SEQUENCE LISTING (6 sequences)

SEQ ID NO: 1 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 2 - protein, 408 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 3 - protein, 538 aa, Homo sapiens
MEESVNQMQPLNEKQIANSQDGYVWQVTDMNRLHRFLCFGSEGGTYYIKEQKLGLENAEALIRLIEDGRGCEVIQEIKSFSQEGRTTKQEPMLFALAICSQCSDISTKQAAFKAVSEVCRIPTHLFTFIQFKKDLKESMKCGMWGRALRKAIADWYNEKGGMALALAVTKYKQRNGWSHKDLLRLSHLKPSSEGLAIVTKYITKGWKEVHELYKEKALSVETEKLLKYLEAVEKVKRTRDELEVIHLIEEHRLVREHLLTNHLKSKEVWKALLQEMPLTALLRNLGKMTANSVLEPGNSEVSLVCEKLCNEKLLKKARIHPFHILIALETYKTGHGLRGKLKWRPDEEILKALDAAFYKTFKTVEPTGKRFLLAVDVSASMNQRVLGSILNASTVAAAMCMVVTRTEKDSYVVAFSDEMVPCPVTTDMTLQQVLMAMSQIPAGGTDCSLPMIWAQKTNTPADVFIVFTDNETFAGGVHPAIALREYRKKMDIPAKLIVCGMTSNGFTIADPDDRGMLDMCGFDTGALDVIRNFTLDMI

SEQ ID NO: 4 - protein, 22 aa, Artificial sequence
  Biotin peptide
ASSLRQILDSQKMEWRSNAGGS

SEQ ID NO: 5 - protein, 196 aa, Escherichia coli
MSVIKMTDLDLAGKRVFIRADLNVPVKDGKVTSDARIRASLPTIELALKQGAKVMVTSHLGRPTEGEYNEEFSLLPVVNYLKDKLSNPVRLVKDYLDGVDVAEGELVVLENVRFNKGEKKDDETLSKKYAALCDVFVMDAFGTAHRAQASTHGIGKFADVACAGPLLAAELDALGKAGGEGKVLPAVAMLEERAKK

SEQ ID NO: 6 - protein, 61 aa, Saccharomyces cerevisiae
MSDSNQGNNQQNYQQYSQNGNQQQGNNRYQGYQAYNAQAQPAGGYYQNYQGYSGYQQGGYQ

(19) United States
(12) Patent Application Publication
(10) Pub. No.: US 2014/0134601 A1
(43) Pub. Date: May 15, 2014

(54) USE OF PROTEIN NANOPARTICLE BASED HYDROGEL

(71) Applicant: KOREA UNIVERSITY RESEARCH AND BUSINESS FOUNDATION, Seoul (KR)

(72) Inventors: Jee Won LEE, Seoul (KR); Eun Jung LEE, Seoul (KR); Jong Hwan LEE, Seoul (KR)

(73) Assignee: KOREA UNIVERSITY RESEARCH AND BUSINESS FOUNDATION, Seoul (KR)

(21) Appl. No.: 13/938,575

(22) Filed: Jul. 10, 2013

(30) Foreign Application Priority Data
Nov. 9, 2012 (KR) 10-2012-0126843

(51) Int. Cl. G01N 33/543; G01N 33/569

(52) U.S. Cl. CPC G01N 33/54346; G01N 33/56988
USPC 435/5; 530/350; 530/387.3; 436/501; 435/7.9; 435/7.92

(57) ABSTRACT

The present invention relates to a use of a protein nanoparticle-based hydrogel, and more particularly, to a use of a protein nanoparticle-based hydrogel capable of highly sensitive and simultaneous multi-detection of disease markers by using a hydrogel to which a protein nanoparticle representing a disease marker detection probe is immobilized.

### CROSS-REFERENCE TO RELATED APPLICATION

This application claims priority to and the benefit of Korean Patent Application No. 2012-0126843, filed on Nov. 9, 2012, the disclosure of which is incorporated herein by reference in its entirety.

### BACKGROUND

1. Field of the Invention

The present invention relates to a use of a protein nanoparticle-based hydrogel capable of highly sensitive and simultaneous multi-detection of disease markers by using a hydrogel to which a protein nanoparticle representing a disease marker detection probe is immobilized.

2. Discussion of Related Art

In protein detecting technology based on specific protein-protein interactions, for example, antigen-antibody, it is important to activate a protein probe and maintain a specific binding capacity to a target molecule. Many conventional methods for attaching proteins to solid substrate surfaces of various protein chips are carried out by simple adsorption/spread or based on immobilization through covalent bond between primary amine groups on proteins. However, typically, a protein is randomly attached to a substrate surface and a structure of the protein is easily modified, so that activity of the protein is inhibited and the protein cannot be bound to a material on the surface, resulting in low efficiency of specific binding. Further, if a protein probe is immobilized on a substrate surface by simple adsorption, the protein probe may be washed away by intensive washing conditions during a detection process, or may be transferred to another molecule having a higher affinity to the substrate surface, and particularly, it may be difficult to quantitatively control the protein probe immobilized on the substrate surface of protein chip and maintain activity of the protein probe [Kusnezow, W. Hoheisel, J. D. J. Mol. Recognit. 16, 165-176 (2003); Park, J. S. et al. Nat. Nanotechnol. 4, 259-264 (2009); Kingsmore, S. F. Nat Rev Drug Discov. 5(4), 310-20 (2006); Ellington, A. A., Kullo, I. J., Bailey, K. R. & Klee, G. G. Clin. Chem. 56(2), 186-193 (2010)].

Unlike conventional organic and inorganic nanoparticles (metal nanoparticles) which are artificially synthesized, protein nanoparticles as nanomaterials synthesized by self-assembly in a cell of a living organism can secure uniform particle size distribution and stability and can be easily mass-produced in a cell of a microorganism. Further, the protein nanoparticles can be developed to have various characteristics/functions by genetically engineered surface modification. In particular, when a disease marker detecting peptide or protein (disease marker detection probe) is represented on a surface, the protein nanoparticles can secure uniform orientation, high-density integration, and structural stability. Thus, the protein nanoparticles have been used as a material of a probe for a highly sensitive diagnostic system [Park, J. S. et al. Nat. Nanotechnol. 4, 259-264 (2009); Seo, H. S. et al. Adv. Funct. Mater. 20, 4055-4061 (2010); Lee, J. H. et al. Adv. Funct. Mater. 20, 2004-2009 (2010); Lee, S. H. et al. The FASEB J. 21, 1324-1334 (2007)].

A hydrogel has a three-dimensional porous structure and can maintain a uniform content of moisture therein, and, thus, the hydrogel has been widely used for analyzing and utilizing proteins. In particular, when the hydrogel forms a polymer through a certain coupling reaction, the hydrogel can form a covalent bond with a material having a specific residue. Thus, the hydrogel has been widely used for immobilizing a functional material. If a protein such as an enzyme is immobilized within a hydrogel, it is possible to maintain activity of the enzyme for a long time [Nolan, J. P. TRENDS in Biotechnology 20, 9-12 (2002)]. However, if only a hydrogel is used as an enzyme support, the hydrogel is swollen by moisture and enzymes are spread out of the hydrogel, and thus stability over time is sharply decreased [Basri, M. et al. J. Appl. Polym. Sci. 82, 1404-1409 (2001)]. Therefore, technology for maintaining activity of a protein enzyme or a protein probe for a long time while immobilizing it in a moistened hydrogel is needed.

Accordingly, in the present invention, among incurable diseases, Sjögren's syndrome and acquired immune deficiency syndrome which cannot be clinically diagnosed from symptoms only are selected as model diseases, a protein nanoparticle representing a detecting probe specific to the two diseases on a surface of the protein nanoparticle is synthesized, and a three-dimensional diagnostic sensor system having maximized surface area and stability is developed by fusing the protein nanoparticle with a three-dimensional porous hydrogel so as to construct a practical diagnostic system capable of highly sensitive and simultaneous multi-detection.

### SUMMARY OF THE INVENTION

The present invention is directed to effectively detect disease markers by using a protein nanoparticle representing a disease marker detection probe on its surface so as to control high-density integration and orientation of the detecting probe, immobilizing the protein nanoparticle to a three-dimensional porous hydrogel so as to remarkably increase a surface area to volume of a diagnostic system, and maintaining activity of the detection probe for a long time and quantitatively controlling the detection probe.

One aspect of the present invention provides a disease marker detection kit including a hydrogel to which a protein nanoparticle representing a disease marker detection probe is immobilized.

Another aspect of the present invention provides a disease marker detection method including: reacting one or more hydrogels to which a protein nanoparticle representing a disease marker detection probe is immobilized with a sample to be detected; reacting a reaction product obtained from the above step with a reporter probe; and detecting one or more disease markers by measuring a change of absorbance or fluorescence intensity in the sample by a bound state of the disease marker-the disease marker detection probe-the reporter probe.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 is a schematic diagram of a spherical protein nanoparticle representing a Sjögren's autoantibody detection probe (La or Ro) or an AIDS marker antibody detection probe (gp41 peptide) as a disease marker detection probe, and an expression vector of the protein nanoparticle;

FIG. 2 provides TEM images of a spherical protein nanoparticle representing a disease marker detection probe, and specifically, FIG. 2(a) shows a protein nanoparticle representing a Ro protein as a Sjögren's autoantibody detection probe; FIG. 2(b) shows a protein nanoparticle representing a La protein as a Sjögren's autoantibody detection probe; and FIG. 2(c) shows a protein nanoparticle representing a La protein as a Sjögren's autoantibody detection probe and an AIDS marker antibody detection probe (gp41 peptide);

FIG. 3 provides a schematic diagram (FIG. 3(a)) showing a manufacturing process of a spherical protein nanoparticle-based hydrogel, a SEM image (FIG. 3(b)) of a manufactured protein nanoparticle-hydrogel complex, and a SEM image (FIG. 3(c)) of distribution of protein nanoparticles immobilized to the hydrogel;

FIG. 4 provides a schematic diagram showing a spherical fluorescent protein nanoparticle immobilized to a two-dimensional substrate and a three-dimensional hydrogel, and a graph showing a change of fluorescence intensity of the fluorescent protein nanoparticle over time;

FIG. 5 is a schematic diagram of a target antibody detection system using a spherical protein nanoparticle-based hydrogel for diagnosing AIDS and Sjögren's syndrome;

FIG. 6 shows sensitivity verification results of a target antibody detection system using a spherical protein nanoparticle-based hydrogel simultaneously representing a La protein and a gp41 peptide for diagnosing AIDS and Sjögren's syndrome, and specifically, FIG. 6(a) shows La autoantibody detection sensitivity verification results and FIG. 6(b) shows AIDS target antibody sensitivity verification results;

FIG. 7 provides sensitivity verification results (FIG. 7(a)) of a spherical protein nanoparticle-based hydrogel at the time of diagnosis of Sjögren's syndrome and sensitivity comparison experiment results (FIG. 7(b)) of a commercial ELISA kit;

FIG. 8 shows emission wavelength measurement results (excitation wavelength: 350 nm) of quantum dots selected to be applied to a simultaneous multi-detection system;

FIG. 9 is a schematic diagram of simultaneous multi-detection of AIDS and Sjögren's syndrome by using a spherical protein nanoparticle-based hydrogel;

FIG. 10 shows results ((a) to (f)) of application of AIDS and Sjögren's syndrome patient samples at the time of simultaneous multi-detection of AIDS and Sjögren s syndrome by using a spherical protein nanoparticle-based hydrogel;

FIG. 11 provides a schematic diagram (FIG. 11(a)) of a protein nanorod expression vector representing a disease marker detection probe [Sjögren's autoantibody detection probe (La)] and an expression result in E. coli (FIG. 11(b));

FIG. 12 provides a schematic diagram showing an assembly process of a protein nanorod representing a disease marker detection probe and TEM images thereof;

FIG. 13 provides a schematic diagram showing a manufacturing process of a protein nanorod fusion hydrogel and SEM images thereof;

FIG. 14 is a schematic diagram of a target antibody detection system using a protein nanorod fusion hydrogel for diagnosing Sjögren's syndrome;

FIG. 15 shows sensitivity verification results of a target antibody detection system using a protein nanorod fusion hydrogel for diagnosing Sjögren's syndrome in a PBS buffer;

FIG. 16 shows sensitivity verification results of a target antibody detection system using a protein nanorod fusion hydrogel for diagnosing Sjögren's syndrome in human serum;

FIG. 17 provides a schematic diagram of a protein nanorod expression vector simultaneously representing a disease marker detection probe [Sjögren's autoantibody detection probe (La)] and biotin, and TEM images thereof;

FIG. 18 is a schematic diagram of a target antibody detection system using a streptavidin-biotin bond-based protein nanorod fusion hydrogel; and

FIG. 19 shows sensitivity verification results of a target antibody detection system using a streptavidin-biotin bond-based protein nanorod fusion hydrogel in a PBS buffer.

### DETAILED DESCRIPTION OF EXEMPLARY EMBODIMENTS

Hereinafter, the present invention will be described in detail with reference to examples and comparative examples. However, the present invention is not limited to these examples.

Hereinafter, a configuration of the present invention will be explained in detail.

The present invention relates to a disease marker detection kit comprising a hydrogel to which a protein nanoparticle representing a disease marker detection probe is immobilized.

In the present specification, the term “recombinant protein or fusion protein” means a protein in which another protein is linked or another amino acid sequence is added to a specific portion, i.e., an N-terminal or a C-terminal, of the protein.

The term “chimeric protein” or “protein nanoparticle probe” is used in the broadest sense to mean a protein or a protein nanoparticle to which various functions are given by bonding a foreign biomaterial to a surface of the protein nanoparticle based on genetic engineering technology or protein engineering technology. Although a human-derived ferritin heavy chain or a Sup35 protein derived from Saccharomyces cerevisiae has been used as a model scaffold for surface-representing a disease marker detection probe, a protein capable of self-assembly, a virus capsid protein, or virus-like particles can be used for forming a chimeric protein, a protein nanoparticle, or a protein nanoparticle probe representing a disease marker detection probe. The protein nanoparticle may have a spherical shape, a rod shape, a linear shape, or the like. If the protein nanoparticle has a spherical shape, a diameter may be in a range of, but not particularly limited to, 5 to 100 nm. The rod-shaped protein nanoparticle can also be used as a “protein nanorod”.

The term “representation” or “expression” is used to represent a foreign protein on a protein nanoparticle surface such as an N-terminal (or an amino terminal) or a C-terminal (or a carboxyl terminal), and while being fused and expressed with a protein capable of self-assembly, the foreign protein can be surface-represented or expressed at the N-terminal or the C-terminal of the protein nanoparticle.

The term “expression vector” refers to a linear or a circular DNA molecule composed of a fragment encoding a target polypeptide operably linked to an additional fragment for transcription of the expression vector. The additional fragment includes a promoter and a stop codon sequence. The expression vector contains one or more replication origins, one or more selection markers, a polyadenylation signal, and the like. The expression vector is generally originated from plasmid or virus DNA or contains elements from both.

Technology for fusing a three-dimensionally structured hydrogel with a protein nanoparticle probe according to the present invention enables high integration and orientation control of a detection probe, and also enables maintenance of activity of the detection probe for a long time and quantitative control together with a significant increase in surface area to volume of a diagnostic system. That is, a protein nanoparticle-based hydrogel has a three-dimensional structure having very uniform porosity and has an excellent ability of moisture maintenance. Thus, modification of the protein nanoparticle is prevented so as to maintain activity for a long time and also uniformly distribute and quantitatively control the protein nanoparticles immobilized in the hydrogel.

According to an exemplary embodiment, when the protein nanoparticle-based hydrogel was used to detect disease markers of Sjögren's syndrome and AIDS, remarkably improved sensitivity could be shown as compared with an existing common diagnosis system. Further, when the protein nanoparticle-based hydrogel was used for an experiment with blood of a Sjögren's syndrome patient and an AIDS patient, disease markers of the two diseases were detected effectively with high stability, sensitivity and specificity.

Such results show that the protein nanoparticle-based hydrogel of the present invention can overcome technical limits (low sensitivity, specificity, reproducibility) of existing diagnosis systems and can be used as a base platform of a highly sensitive and simultaneous multi-detection nanobiosensor, and can also be used as a highly sensitive diagnosis system for blood of patients.

In a disease marker detection kit according to the present invention, a disease marker may be an autoantibody of a human autoimmune disease such as an anti-La autoantibody or an anti-Ro autoantibody of Sjögren's syndrome or an anti-virus antibody of a viral disease such as an HIV-1 anti-gp41 antibody, but is not limited thereto.

The disease marker detection probe may vary depending on a kind of a disease marker and is not particularly limited. For example, the disease marker detection probe may be a protein or a peptide which can be bound to a disease marker, or an antibody. The protein or peptide which can be bound to a disease marker may use one or two or more antigen proteins specific to autoantibodies of human autoimmune diseases, such as a RO (SSA) protein, a human La (SSA) protein, or virus-derived antigen proteins or peptides such as an HIV-1 gp41 peptide.

The protein nanoparticle representing the disease marker detection probe may be manufactured from a chimeric protein fused with a protein capable of self-assembly and one or more disease marker detection probes.

An expression vector containing the chimeric protein can be introduced into a host cell so as to be transformed, and a target transformant can be selected by using an antibiotic marker.

The selected transformant can be cultured by a typical culture method and then purified, so that a protein nanoparticle of the present invention can, be obtained.

The transformation may include any method of introducing a nucleic acid into an organism, a cell, a tissue, or an organ, and can be carried out by selecting standard technology appropriate for a host cell that is known in the art. The methods may include electroporation, protoplast fusion, calcium phosphate (CaPO₄) precipitation, calcium chloride (CaCl₂) precipitation, stirring using silicon carbide fibers, agrobacteria-mediated transformation, PEG, dextran sulfate, lipofectamine, etc., but are not limited thereto.

An expression amount and post-translational modifications of a protein may vary depending on a kind of a host cell, and, thus, a host cell most suitable for a purpose may be selected and used.

The host cell may include a prokaryotic host cell such as Escherichia coli, Bacillus subtilis, Streptomyces, Pseudomonas, Proteus mirabilis, or Staphylococcus, but is not limited thereto. Further, the host cell may use lower eukaryotic cells such as mycete (for example, Aspergillus), yeast (for example, Pichia pastoris, Saccharomyces cerevisiae, Schizosaccharomyces, Neurospora crassa), and cells derived from higher eukaryotes including insect cells, plant cells, mammal cells, etc.

In the protein nanoparticle-based hydrogel, the protein nanoparticle is immobilized to the hydrogel through a covalent bond, and, thus, it is possible to improve upon loss of a disease marker detection probe.

A protein nanoparticle representing such a disease marker detection probe can be prepared by modifying a protein nanoparticle into a chemical structure which can be polymerized, and then reacting it with a polymer precursor for preparing a hydrogel.

To be more specific, the protein nanoparticle can be prepared by a polymerization reaction between a protein nanoparticle expressed by Chemical Formula 1 below and a polymer precursor solution:

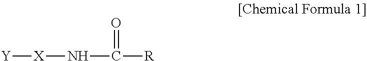

In Chemical Formula 1, X represents a protein nanoparticle. Y represents a disease marker detection probe, and R represents a vinyl group, an acryl group, or an acryl group substituted or not substituted by an alkyl having 1 to 30 carbon atoms.

The terms used for defining substituents of compounds of the present invention are as follows.

The term “alkyl” refers to a linear, branched, or cyclic saturated hydrocarbon having 1 to 30 carbon atoms, unless context dictates otherwise. A C₁₋₃₀ alkyl group may include, for example, but is not limited to, methyl, ethyl, propyl, butyl, pentyl, hexyl, heptyl, octyl, nonyl, decyl, isopropyl, isobutyl, sec-butyl and tert-butyl, isopentyl, neopentyl, isohexyl, isoheptyl, isooctyl, isononyl, and isodecyl. Further, the alkyl may include “cycloalkyl”. The cycloalkyl refers to a non-aromatic saturated hydrocarbon ring having 3 to 12 carbon atoms and includes a mono ring and a fusion ring, unless context dictates otherwise. A representative example of a C₃₋₁₂ cycloalkyl may include, but is not limited to, cyclopropyl, cyclobutyl, cyclopentyl, cyclohexyl, cycloheptyl, and cyclooctyl.

In Chemical Formula 1 above, the substituent group R means a functional group which can be polymerized with a polymer, and a terminal amine group of the protein nanoparticle is substituted by the functional group so as to react with a polymer precursor for preparing a hydrogel.

Therefore, the substituent group R may represent a vinyl group, an acryl group, or an acryl group substituted or not substituted by an alkyl having 1 to 30 carbon atoms. To be more specific, the substituent group R may represent a vinyl group, an acryl group, or an acryl group substituted or not substituted by an alkyl having 1 to 6 carbon atoms. To be most specific, the substituent group R may represent a vinyl group.

According to an exemplary embodiment, an amine group represented on a surface of a protein nanoparticle reacts with N-succinimidylacrylate (NSA) so as to prepare a protein nanoparticle having a vinyl group which can be polymerized. The compound of Chemical Formula 1 refers to such a surface-modified protein nanoparticle.

The polymer may use one or two or more of polyacrylic acid, polyacrylamide, polyhydroxyethyl methacrylate, polyethyleneglycol, poly(N,N-ethylaminoethyl methacrylate), hyaluronic acid, or chitosan.

The polymer precursor solution may be prepared by adding a polymer to water or a buffer solution. As the buffer solution, PBS (Phosphate Buffered Saline) or the like may be used.

Basically, gelation of a polymer precursor solution is a polymerization process of a mixture of monomers of the polymer and can be carried out by a chemical polymerization method in which the reaction is carried out by chemical breakdown of a polymerization initiator, or a photochemical polymerization method in which the reaction is carried out by a photoinitiator such as UV or plasma.

The polymer precursor solution may further contain a polymerization initiator in an amount of 0.1 to 0.2 parts by weight with respect to 100 parts by weight of the polymer.

The polymerization initiator may use one or two or more of ammonium persulfate, tetramethylethylenediamine, riboflavin, riboflavin-5′-phosphate, 2-hydroxy-2-methylpropanon, or 2,2-diethoxyacetophenone.

A disease marker detection kit of the present invention may further include a reporter probe which can be bound to a complex of a disease marker and a disease marker detection probe.

The reporter probe is configured to detect a bound form of the disease marker and the disease marker detection probe. For example, if the disease marker is an anti-La autoantibody or an anti-Ro autoantibody of Sjögren's syndrome, or an HIV-1 anti-gp41 antibody, the disease marker detection probe may be a La, Ro, or gp41 antigen. Therefore, the reporter probe detects an antigen-antibody complex, and the reporter probe can compare an amount of the complex formed and determine existence or nonexistence of a disease marker, an amount of a disease marker, and an existence pattern, and can ultimately diagnose whether a disease has been contracted or not.

Herein, the term “antigen-antibody complex” means a combination of a proteinous disease marker and an antibody specific to the proteinous disease marker or an antibody. Typically, an amount or a formation pattern of the antigen-antibody complex formed can be measured by detecting amplitude and a pattern of a signal of a detection label connected with a secondary antibody. Such a detection label may be an enzyme, a fluorescent material, a ligand, a luminescent material, a microparticle, a redox molecule, a radioactive isotope, or the like, but is not necessarily limited thereto. If an enzyme is used as the detection label, available enzymes may include β-glucuronidase, β-D-glucosidase, β-D-galactosidase, urease, peroxidase or alkaline phosphatase, acetylcholinesterase, glucose oxidase, hexokinase and GDPase, RNase, glucose oxidase, luciferase, phosphofructokinase, phosphoenolpyruvate carboxylase, aspartate aminotransferase, and phosphenolpyruvate decarboxylase, β-latamase, etc., but are not limited thereto. If a fluorescent material is used as the detection label, the fluorescent material may include a fluorescent protein, Dylight 488 NHE-ester dye, Vybrant™ DiI, Vybrant™ DiO, a quantum dot nanoparticle, fluorescein, rhodamine, Lucifer yellow, B-phitoerithrin, 9-acridine isothiocyanate, Lucifer yellow VS, 4-acetamido-4′-isothio-cyanatostilbene-2,2′-disulfonic acid, 7-diethylamino-3-(4′-isothiocyatophenyl)-4-methylcoumarin, succinimidyl-pyrenebutyrate, 4-acetamido-4′-isothiocyanatostilbene-2,2′-disulfonic acid derivatives, LC™-Red 640, LC™-Red 705, Cy5, Cy5.5, resamine, isothiocyanate, erythrosine isothiocyanate, diethyltriamine pentaacetate, 1-dimethylaminonaphthyl-5-sulfonate, 1-anilino-8-naphthalene sulfonate, 2-sotitoluidinyl-6-naphthalene sulfonate, 3-phenyl-7-isocyanatocoumarin, 9-isothiocyanatoacridine, acridine orange 9-i(soti(2-benzoxazolyl)phenyl)maleimide sadiazol, stilbene, pyrene, derivatives thereof, fluorescent material-containing silica, semiconductor quantum dots of Groups II and IV, semiconductor quantum dots of Groups III and V, semiconductor quantum dots of Group IV, or mixtures of two or more thereof. Preferably, the fluorescent material may include one or more selected from the group consisting of a quantum dot nanoparticle, Cy3.5, Cy5, Cy5.5, Cy7, ICG (indocyanine green), Cypate, ITCC, NIR820, NIR2, IRDye78, IRDye80, IRDye82, Cresy Violet, Nile Blue, Oxazine 750, Rhodamine800, the lanthanide series, and Texas Red. If the fluorescent material is the quantum dot nanoparticle, compounds of Groups II to VI or Groups III to V may be used. In this case, the quantum dot nanoparticle may include one or more selected from the group consisting of CdSe, CdSe/ZnS, CdTe/CdS, CdTe/CdTe, ZnSe/ZnS, ZnTe/ZnSe, PbSe, PbS, InAs, InP, InGaP, InGaP/ZnS, and HgTe. If a ligand is used as the detection label, available ligands may include biotin derivatives and the like, but are not limited thereto. If a luminescent material is used as the detection label, available luminescent materials may include acridinium ester, luciferin, luciferase, etc., but are not limited thereto. If a microparticle is used as the detection label, available microparticles may include colloid gold, tinted latex, etc., but are not limited thereto. If a redox molecule is used as the detection label, available redox molecules may include ferrocene, a ruthenium complex compound, viologen, quinone, Ti ions, Cs ions, diimide, 1,4-benzoquinone, hydroquinone, K₄W(CN)₈, [Os(bpy)₃]²⁺, [RU(bpy)₃]²⁺, [MO(CN)₈]⁴⁻, etc., but are not limited thereto. If a radioactive isotope is used as the detection label, available radioactive isotopes may include ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, or ¹⁸⁶Re, but are not limited thereto.

For example, the reporter probe may be any one of an anti-human IgG conjugated with a reporter enzyme such as HRP (Horseradish Peroxidase) or AP (Alkaline Phosphatase); a virus antigen such as an HIV-1 gp41 peptide; a biotin-bound virus antigen such as a biotin-bound HIV-1 gp41 peptide; or a human autoimmune antigen such as a biotin-bound human La (SSA) protein or Ro (SSA) protein, but is not limited thereto since it may vary depending on a kind of a disease marker.

The present invention also relates to a disease marker detection method including: reacting one or more hydrogels to which a protein nanoparticle representing a disease marker detection probe is immobilized with a sample to be detected; reacting a reaction product obtained from the above step with a reporter probe; and detecting one or more disease markers by measuring a change of absorbance or fluorescence intensity in the sample by a bound state of the disease marker-the disease marker detection probe-the reporter probe.

In the present invention, a hydrogel to which a protein nanoparticle representing a disease marker detection probe is bound can react with a disease marker and show a change of absorbance or fluorescence, so that one or more disease markers can be detected by measuring a change of absorbance or fluorescence intensity.

In order to detect a single disease marker or multiple disease markers, a hydrogel to which a protein nanoparticle representing one or more disease marker detection probes is immobilized, or one or more hydrogels to which a protein nanoparticle representing a disease marker detection probe is immobilized, may be used so as to react with a sample.

The disease marker may be an autoantibody of a human autoimmune disease such as an anti-La autoantibody or an anti-Ro autoantibody of Sjögren's syndrome, or an anti-virus antibody of a viral disease such as an HIV-1 anti-gp41 antibody, but is not limited thereto.

The disease marker detection probe may be a protein or a peptide which can be bound to a disease marker, or an antibody. For example, the disease marker detection probe may include, but is not particularly limited to, antigen proteins specific to autoantibodies of human autoimmune diseases such as a human RO(SSA) protein, a human La(SSA) protein, or virus-derived antigen proteins such as an HIV-1 gp41 peptide.

The protein nanoparticle representing the disease marker detection probe may be prepared by the above-described method. For example, the protein nanoparticle can be prepared from any one protein capable of self-assembly among a ferritin heavy chain, a Sup35 protein derived from Saccharomyces cerevisiae, or a virus capsid protein, and a chimeric protein fused with one or more disease marker detection probes. In this case, the disease marker and the disease marker detection probe may be proteins or peptides.

The hydrogel to which the protein nanoparticle representing the disease marker detection probe is immobilized may be prepared by making a reaction between a protein nanoparticle and a polymer precursor solution, and a kind of the polymer and a polymerization method may be the same as described in the above method.

As the sample to be detected, a biological sample of a subject may be used and may include, for example, a tissue, a cell, whole blood, serum, blood plasma, saliva, cerebrospinal fluid, urine, etc.

Since the reporter probe is configured to detect a bound form of the disease marker and the disease marker detection probe, the reporter probe may be the same one as described above, but is not particularly limited thereto as long as it can be combined with the disease marker.

If the disease marker is an antibody, the disease marker detection probe may be an antigen and the reporter probe detects the antigen-antibody complex, and an amount or a pattern of the complex formed may be analyzed by, but is not limited to analysis by, western blot, ELISA (enzyme linked immunosorbent assay), radioimmunoassay, radioimmunodiffusion, an Ouchterlony technique, rocket immunoelectrophoresis, immunohistologic staining, immunoprecipitation assay, complement fixation assay, FACS, a protein chip assay, etc.

Through the above analysis methods, it is possible to compare an amount of an antigen-antibody complex formed in a biological sample of a normal person with an amount of an antigen-antibody complex formed in a biological sample of a suspected Sjögren's syndrome or AIDS patient, so that it is possible to determine existence or nonexistence of a proteinous disease marker for diagnosing Sjögren's syndrome or AIDS, an amount and a pattern thereof, and it is ultimately possible to diagnose whether or not Sjögren's syndrome or AIDS has been contracted by the suspected Sjögren's syndrome or AIDS patient in early stages.

Hereinafter, the present invention will be described in further detail with respect to examples according to the present invention and comparative examples not according to the present invention, but the scope of the present invention is not limited by the following examples.

### EXAMPLE 1

### Manufacturing of Spherical Protein Nanoparticle-Based Hydrogel

(Manufacturing Expression Vector for Synthesis of Spherical Protein Nanoparticle Representing Disease Marker Detection Probe)

The present inventors selected Sjögren's syndrome and acquired immune deficiency syndrome (AIDS) as model diseases. It is known that these two diseases have different causes but similar symptoms. It is known that AIDS is caused by infection with human immunodeficiency virus (HIV) and serum of an AIDS patient contains various marker antibodies that recognize the HIV as an antigen. In particular, HIV-1 gp41 is an immunodominant region recognized by an antibody. It is known that most AIDS patients have an anti-gp41 antibody. A diagnosis system using a part of this antigen as a detection probe was developed. It is known that if a person is infected with HIV, his/her symptoms may escalate into symptoms (rheumatological manifestation) similar to those of an autoimmune disease patient. It is known that as for a DILS (diffuse infiltrative lymphocytosis syndrome; Sjögren-like syndrome) patient having symptoms such as xerophthalmia or xerostama, which are very similar to symptoms of Sjögren's syndrome, it is difficult to make a clinical diagnosis based on symptoms only. If Sjögren's syndrome is not treated, it can lead to life-threatening complications, such as angitis or invasion into kidneys, lungs, or the entire body. AIDS caused by infection with virus is a high-risk infectious disease, and if AIDS cannot be diagnosed in its early stages, it can be spread. Therefore, these two diseases must be distinguished and confirmed in their early stages. The biggest difference between DILS and SS is that anti-Ro and anti-La autoantibodies do not exist in serum of a DILS patient. Further, detection of anti-Ro and anti-La autoantibodies has been used during a clinical diagnosis of SS.

Based on the above description, the present inventors selected a Sjögren's syndrome autoantibody detection probe (La protein or Ro protein) or an AIDS marker antibody detection probe (gp41 peptide) as a disease marker detection probe, manufactured a production vector by inserting the Sjögren's syndrome autoantibody detection probe (La protein or Ro protein) or AIDS marker antibody detection probe (gp41) gene into a carboxyl terminal of a protein nanoparticle, and expressed the production vector in E. coli.

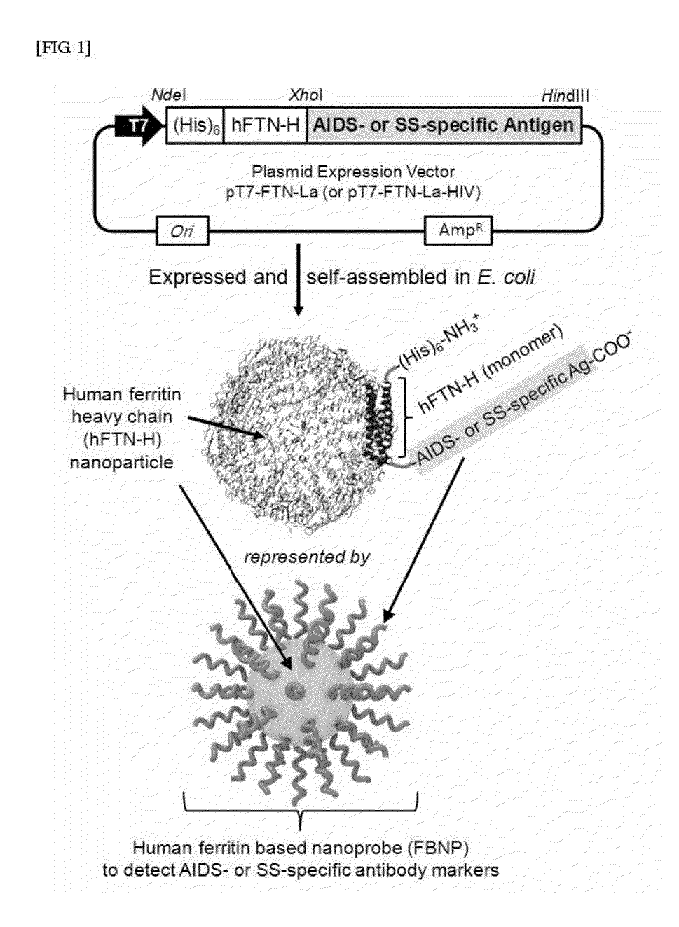

In order to do so, gene clones for coding NH₂-NdeI-hexahistidine-[human-derived ferritin heavy chain (SEQ ID NO:1)]-XhoI-COOH and NH₂-XhoI-[human-derived La protein (SEQ ID NO:2)]-HindIII-COOH (or NH₂-XhoI-[human-derived Ro protein (SEQ ID NO:3)]-HindIII-COOH or NH₂-XhoI-[human-derived La protein]-BamHI-COOH and NH₂-BamHI-[gp41 peptide]-[gp41 peptide]-HindIII-COOH) were PCR amplified by using an adequate primer and ligated to an NdeI-XhoI-BamHI-HindIII cloning site of pT7-7, so that an expression vector pT7-FTNH-La (or pT7-FTNH-Ro or pT7-FTNH-La-gp41-gp41) for coding synthesis of a recombinant ferritin protein nanoparticle representing a disease marker detection probe (La or Ro or gp41) on its surface (FIG. 1) was manufactured. All manufactured plasmid expression vectors were gelated and purified, and then sequences thereof were checked by complete DNA sequencing. Further, a sequence of the gp41 peptide was used as described in Nelson J. D. et al. J. Virol. 81, 4033-4043 (2007).

(Manufacturing Expression Vector for Biosynthesis of Biotin Fusion Reporter Probe)

Gene clones for coding NH₂-NdeI-hexahistidine-[biotin peptide (SEQ ID NO:4)]-[linker (G3SG3TG3SG3)]-[human-derived La protein]-HindIII-COOH (or NH₂-NdeI-hexahistidine[N-ePGK (N-terminal domain of E. coli phosphoglycerate kinase)]-[biotin peptide]-[linker (G3SG3TG3GS3)]-[human-derived Ro protein]-HindIII-COOH fused with N-ePGK (SEQ ID NO: 5) as a fusion tag developed by the present inventors for water-soluble expression of a Ro protein, if alone, showing insoluble expression in E. coli) were PCR amplified by using an adequate primer and ligated to an NdeI-HindIII cloning site of pT7-7, so that an expression vector pT7-biotin-La (or pT7-NePGK-biotin-Ro) for coding a biotin fusion reporter probe (La or Ro or gp41) was manufactured. All manufactured plasmid expression vectors were gelated and purified, and then sequences thereof were checked by complete DNA sequencing.

(Manufacturing and Expression of Transformant for Biosynthesis of Protein Nanoparticle Representing Disease Marker Detection Probe and Reporter Probe)

The vectors manufactured above by a method described by Hanahan (Hanahan D, DNA Cloning vol. 1 109-135, IRS press 1985) were transformed in E. coli.

To be specific, the vectors manufactured above were transformed by a thermal shock method in E. coli BL21 (DE3) treated with CaCl₂ and then cultured in a culture medium containing ampicillin, so that a colony which was transformed from the expression vector and had ampicillin resistance was sorted. A part of a seed culture solution obtained by culturing the colony in a LB culture medium for overnight was introduced into a LB culture medium containing 100 mg/mL ampicillin and then cultured at 37° C. at 130 rpm. When OD₆₀₀ of the culture solution reached 0.7 to 0.8, IPTG (0.5 mM) was added and a temperature was lowered to 20° C. so as to induce an expression of a recombinant gene. After the IPTG was added, the culture solution was additionally cultured for 16 to 18 hours under the same conditions. As for a reporter probe fused with a biotin peptide at an amino terminal, when the IPTG was added, 10 μg/mL of biotin was added to the culture medium so as to be cultured.

(Purification of Protein Nanoparticle Representing Disease Marker Detection Probe and Reporter Probe)

In order to purify a recombinant protein, the E. coli cultured above was collected and its cell pellets were re-suspended in 5 mL of a lysis buffer [pH 8.0, 1 mM PMSF (phenylmethylsulfonyl fluoride: serine proteinase inhibitor), 10% glycerol, 0.1% Triton X-100, 2mM MgCl₂, 50mM Tris-Cl, 0.1 mg/mL lysozyme] and stirred at normal temperature for 15 to 30 minutes and then disrupted by using a sonicator. A cytosol of the disrupted cells was centrifuged at 13,000 rpm for 10 minutes so as to separate a supernatant. Then, each recombinant protein was separated by using a Ni²⁺-NTA column (Qiagen, Hilden, Germany) (washing buffer: pH 8.0, 50 mM sodium phosphate, 300 mM NaCl, 80 mM imidazole; elution buffer: pH 8.0, 50 mM sodium phosphate, 300 mM NaCl, 250 mM imidazole). In order to remove the imidazole from the elution buffer, the buffer was substituted by a PBS buffer by using a membrane filterator (Amicon, 10K).

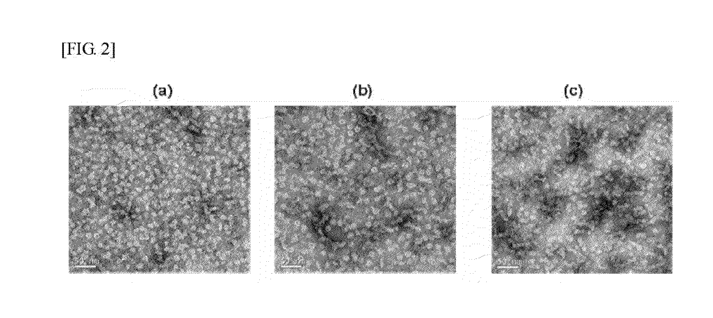

It was confirmed from TEM images of FIG. 2 that uniform spherical nanoparticles were formed.

(Manufacturing Fluorescent Material-Labeled Reporter Probe)

A fluorescent material (quantum dot) as a label was bound to the biotin fused reporter probe. To manufacture a final fluorescent reporter probe, the quantum dot having a surface to which streptavidin was immobilized and the reporter probe fused with biotin at an amino terminal were bound to each other through a biotin-streptavidin bond.

A Ro protein or a La protein (0.05 pmol) fused with biotin at an amino terminal and Quantum dot 565 (5 pmol) having a surface to which streptavidin was immobilized were cultured in a PBS buffer at 25° C. for 2 hours so as to be bound to each other. Then, a reporter probe which was not labeled with the quantum dot was removed by streptavidin affinity chromatography and ultrafiltration (Amicon Ultra 100K).

Since a length of a peptide was too short, a reporter probe (biotin: gp41) for detecting AIDS was manufactured by peptide synthesis and then bound to Quantum dot 800 by the same method as described above.

(Manufacturing Hydrogel to Which Protein Nanoparticle is Immobilized)

10 mg of the protein nanoparticle manufactured above and 0.01 mg of N-succinimidylacrylate (NSA) were incubated in a PBS buffer at 37° C. for 1 hour and bound to each other. Then, non-bound NSA was removed by ultrafiltration (Amicon Ultra 100K), so that a protein nanoparticle having a polymerizable chemical structure was finally manufactured.

According to the records, a polyacrylamide fusion hydrogel has advantages of high stability, low non-specific bonding, low fluorescent background, and the like.

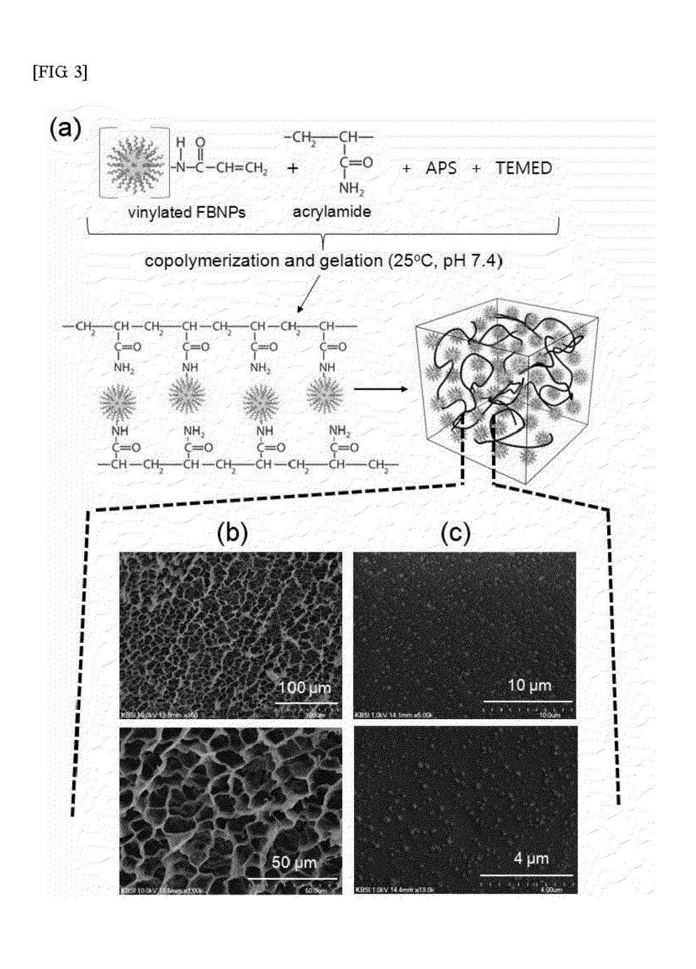

Therefore, 30 μg of the modified protein nanoparticle and 0.9% polyacrylamide (29:1 W/W acrylamide: bis-acrylamide) were mixed in the presence of 0.125% w/v ammonium persulfate (APS) and 0.125% w/v tetramethylethylenediamine (TEMED) and each 50 μl of the mixture was apportioned into a 96-well plate and polymerized at 25° C. for 16 hours, so that a protein nanoparticle-based hydrogel was manufactured (FIG. 3(a)).

As a result, it was confirmed from SEM images that the protein nanoparticle-based hydrogel had a three-dimensional structure having very uniform porosity (FIG. 3(b)).

Further, an amount of detection probes immobilized to a substrate is a key factor for determining sensitivity of a detection system. Thus, in order to construct a highly reliable diagnosis system, technology for uniformly immobilizing detection probes of high density is demanded. Typically, if detection probes are simply adsorbed and immobilized to a two-dimensional surface of a substrate, it is difficult to quantitatively control an actual amount of detection probes immobilized to the substrate. However, as for a protein nanoparticle-based hydrogel, it is possible to control an amount of protein nanoparticles during a manufacturing process and also possible to quantitatively measure an accurate amount of detection probes present on a substrate since the protein nanoparticles are fused and immobilized to the hydrogel.

Therefore, in order to check whether the protein nanoparticles were uniformly dispersed and immobilized to the hydrogel, the protein nanoparticle-based hydrogel representing biotin was reacted with an anti-biotin antibody bound to gold nanoparticles (20 nm) and then the gold nanoparticles were amplified by using a silver enhancement kit.

When SEM images were captured, positions of the protein nanoparticles in the hydrogel could be clearly confirmed through the gold nanoparticles. As shown in FIG. 3(c), it was confirmed that the gold nanoparticles were uniformly dispersed in the hydrogel, which showed that the protein nanoparticles were uniformly dispersed and immobilized throughout the whole area of the hydrogel.

In order to commercialize a disease diagnosis system, it is necessary to maintain stability of a protein detection probe immobilized to a substrate for a long time. Typically, a protein immobilized to a two-dimensional surface is likely to be exposed to air and cannot maintain moisture if it is not treated with a stabilizer, and, thus, it is difficult to preserve the protein for a long time.

In order to verify a protein preservation ability of the protein nanoparticle-based hydrogel constructed by the present inventors, an enhanced green fluorescent protein (eGFP) and protein nanoparticles were fused and expressed and immobilized to each of a two-dimensional polystyrene (PS) surface and a three-dimensional hydrogel-based structure widely used as protein immobilizing substrates. After being filled with nitrogen, they were kept at 25° C., and changes of fluorescence intensity over time were compared.

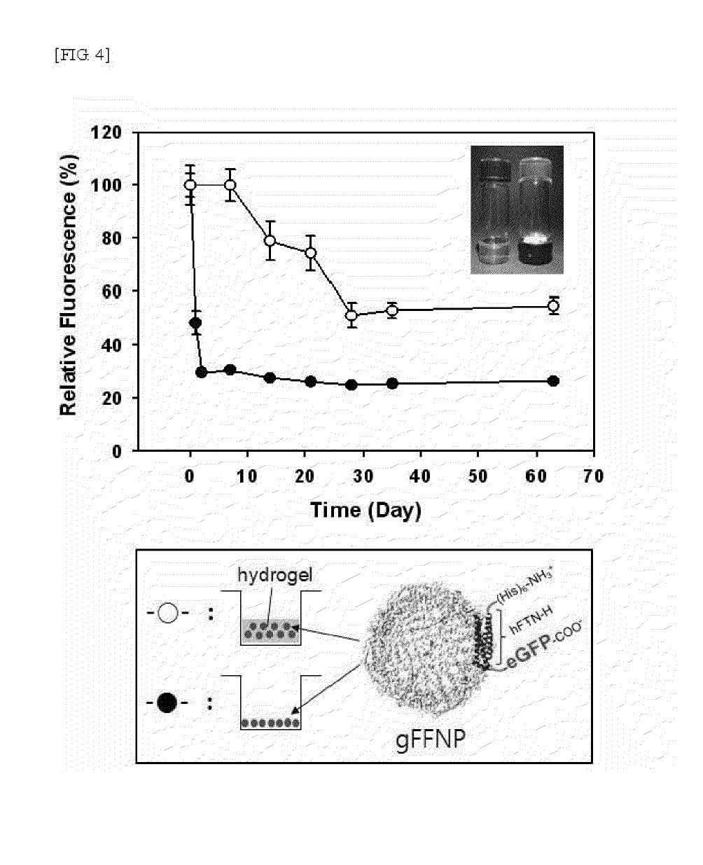

As a result, fluorescence intensity of the fluorescent nanoparticles immobilized to the two-dimensional polystyrene surface decreased to 30% of initial fluorescence intensity after one day, whereas fluorescence intensity of the fluorescent nanoparticles fused and immobilized to the hydrogel only decreased to 50% of the initial fluorescent intensity after a entire month. It is deemed that since the hydrogel has an excellent ability of moisture maintenance as compared with the two-dimensional surface, denaturation of the protein was minimized. This shows that the hydrogel has a great advantage as a detection probe immobilization platform of a practicable diagnosis system to be constructed in the future (FIG. 4).

(Sensitivity Examination of Protein Nanoparticle-Immobilized Hydrogel Fusion Material)

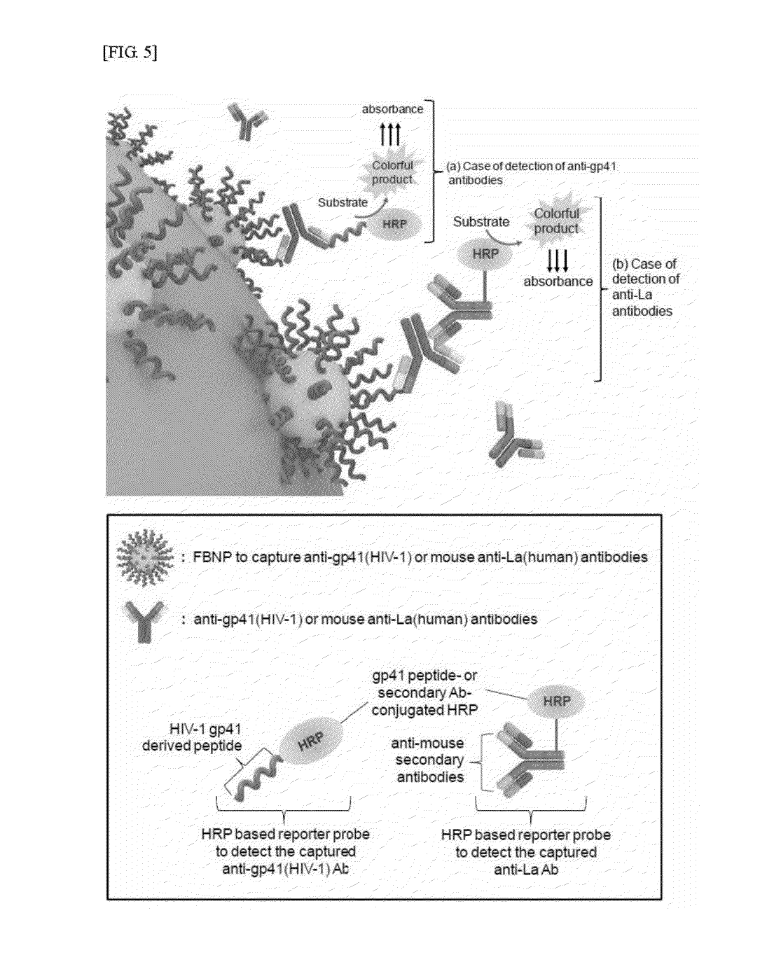

In order to evaluate the utility and performance of the three-dimensional protein nanoparticle-based hydrogel diagnosis system manufactured above, a sensitivity analysis was carried out. The sensitivity analysis was carried out by using anti-gp41 and anti-La antibodies. As shown in FIG. 5, each hydrogel fused with a protein nanoparticle detection probe was reacted with a sample (an anti-gp41 or anti-La antibody or a patient blood sample), and then absorbance thereof was measured by using a secondary antibody bound to a HRP.

Further, the results were compared and analyzed with a typically commercialized ELISA (Enzyme-linked Immunosorbent Assay) kit for blood diagnosis and a diagnosis system in which protein nanoparticles are immobilized to a two-dimensional polystyrene (PS) surface.

In order to do so, the hydrogel containing protein nanoparticles (([FTNH-La-(gp41)₂]) manufactured above was washed by using a PBS buffer, and a PBS buffer containing 100 μl of human serum (serum of Sjögren's syndrome patient or healthy serum) or a mouse anti-La antibody or a human anti-gp41 antibody was added and cultured at normal temperature for 2 hours with stirring. After the hydrogel was washed by using the PBS buffer, 100 μl of “enzyme conjugate reagent [anti-mouse IgG or gp41 peptide conjugated with a HRP enzyme (“enzyme conjugate reagent” was used for comparison with the ELISA kit)]” was added to each well and cultured at normal temperature for 1 hour with stirring and then washed with the PBS buffer. 100 μl of “TMB reagent (containing a substrate to the HRP enzyme)” was added to each well and cultured at normal temperature for 15 minutes and 100 μl of 1M sulfuric acid was added to each well and mixed for 30 sec to stop an enzyme reaction, and then absorbance thereof was measured at 450 nm by using a microplate reader.

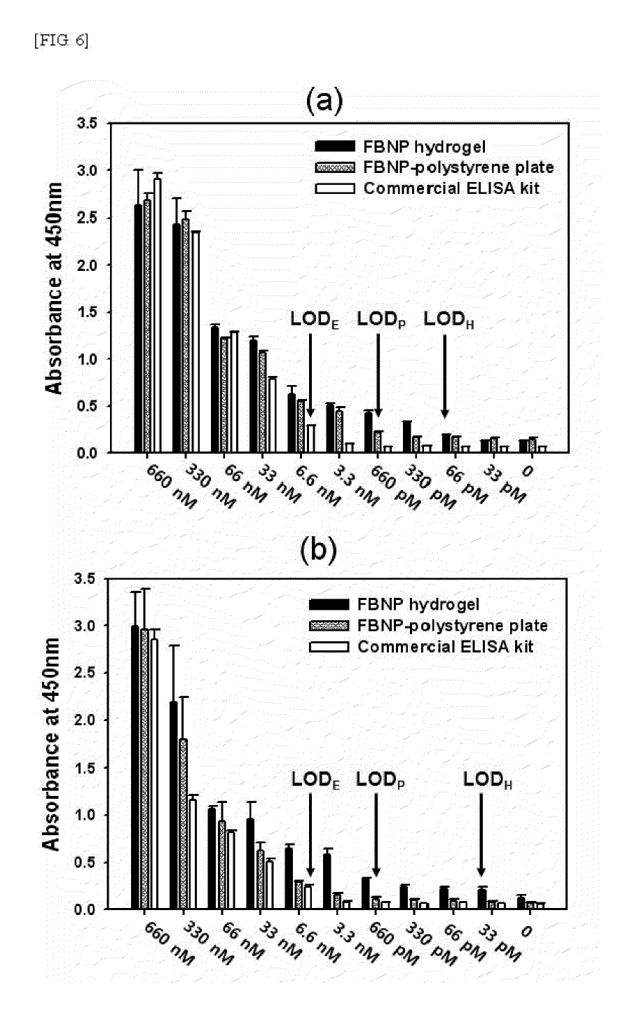

As a result, the two-dimensional ELISA kit showed that the anti-La antibody and the anti-gp41 antibody had LODs [limit of detection: determined as defined by IUPAC (International Union of Pure and Applied Chemistry)] of 6.6 nM as an antibody concentration, whereas the protein nanoparticle-based hydrogel showed that the anti-La antibody and the anti-gp41 antibody had LODs (limit of detection) of 66 pM and 33 pM, respectively, as an antibody concentration (FIG. 6). That is, the protein nanoparticle-based hydrogel showed sensitivity 100 to 200 times higher than the two-dimensional ELISA kit, which showed the excellence of the protein nanoparticle-based hydrogel.

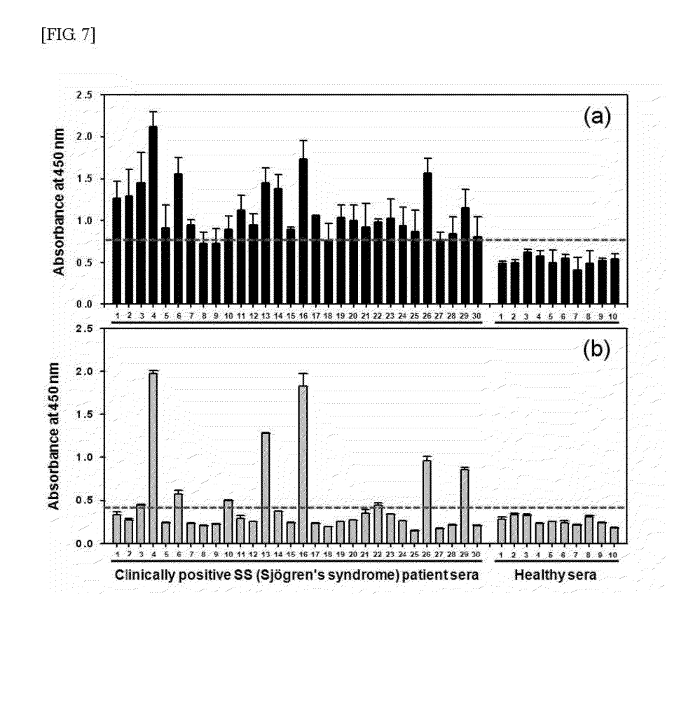

Further, as a result of a comparative experiment with a commercialized ELISA kit for blood diagnosis by using blood samples of Sjögren's syndrome patients, the ELISA kit detected an anti-La antibody from only nine out of thirty patients, resulting in a sensitivity of 30%, whereas the protein nanoparticle-based hydrogel diagnosis system constructed by the present inventors detected an anti-La antibody from twenty six patients, resulting in remarkably high sensitivity of 87% (FIG. 7). It is deemed that since the protein nanoparticle of the present invention is immobilized to the three-dimensional porous hydrogel, it is present in a liquid phase-like condition rather than a solid phase-like condition, which prevents denaturation of the samples and allows easy accessibility, and which shows that the hydrogel has a great advantage as a probe immobilization platform of a diagnosis system.

(Simultaneous Multi-Detection of Disease Using Protein Nanoparticle-Immobilized Hydrogel Fusion Material)

A simultaneous multi-detection system is advantageous in that various diseases can be diagnosed at the same time through one analysis, which is time and cost effective, and diseases can be diagnosed from a small blood sample. However, the simultaneous multi-detection system has drawbacks of low reproducibility and non-specific cross-reactivity which need to be solved.

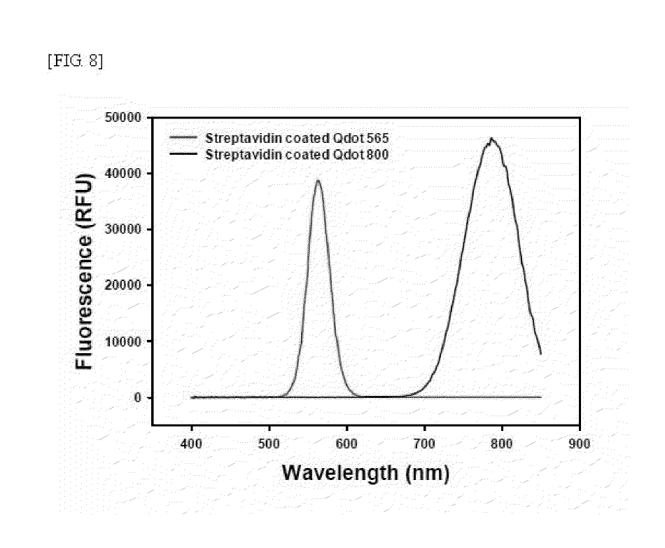

In order to construct a simultaneous multi-detection system for two diseases, the present inventors manufactured a hydrogel by mixing protein nanoparticles containing both a gp41 peptide and a La protein with protein nanoparticles containing a Ro protein as described above, and immobilized a biotin fused La protein or Ro protein to Quantum dot 800-streptavidin as a fluorescent material and immobilized a biotin fused gp41 peptide to quantum dot 585-streptavidin so as to be used as reporter probes for simultaneous detection (Quantum dot 800 and Quantum dot 585 have excitation wavelengths and emission wavelengths which do not overlap with each other, and, thus, they can be used at the same time; see FIG. 8). An experiment was carried out as shown in FIG. 9, and a blood sample of a Sjögren's syndrome patient and a blood sample of an HIV-1 positive patient were mixed and used as shown in FIG. 9.

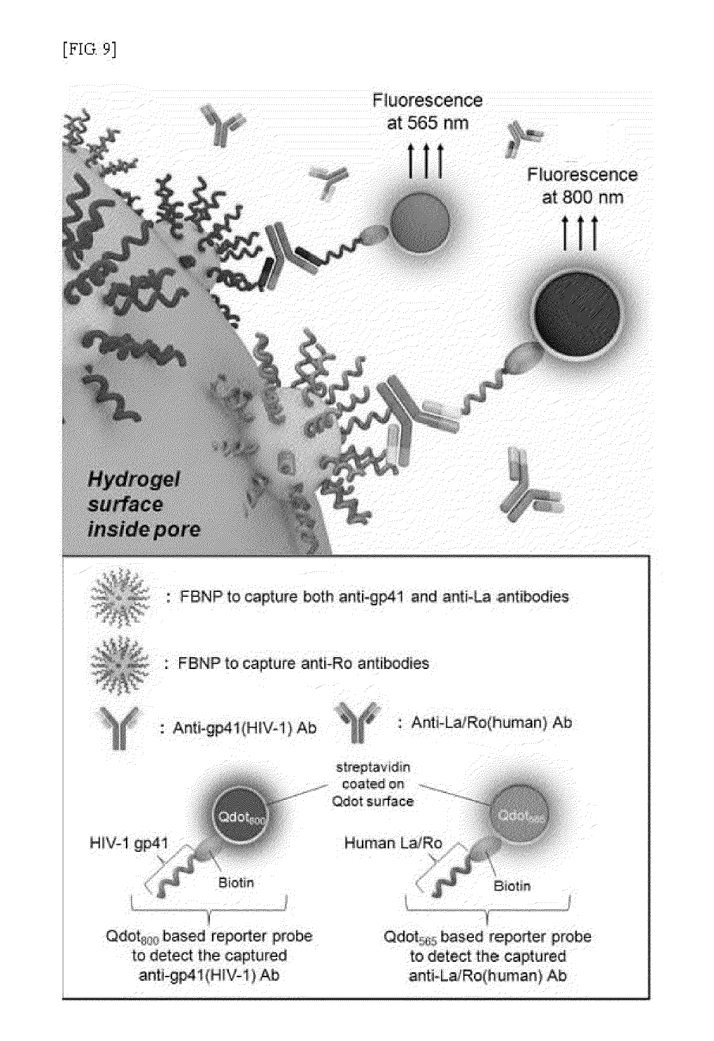

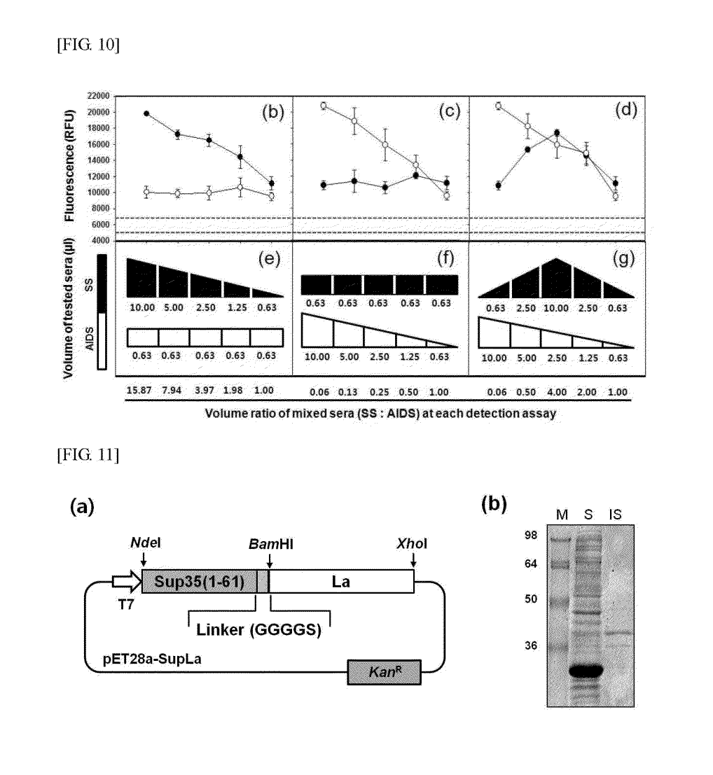

To be more specific, the manufactured hydrogel containing 15 μg of [FTNH-La-(gp41)₂] and 15 μg of [FTNH-Ro] as protein nanoparticles was washed by using a PBS buffer, and 100 μl of a serum mixed solution in which a serum sample of the Sjögren's syndrome patient and the blood sample of the HIV-1 positive patient were mixed as shown in FIGS. 10(d) to 10(f) was added and cultured at normal temperature for 2 hours with stirring. After the hydrogel was washed by using the PBS buffer, 100 μl of the fluorescent reporter probe manufactured above was added to each well and cultured at normal temperature for 1 hour with stirring and then washed with the PBS buffer. Thereafter, fluorescence intensity thereof was measured at an excitation wavelength of 350 nm and an emission wavelength of 565 nm or 800 nm by using a microplate reader.

As shown in FIGS. 10(a) to 10(c), a detection signal was proportional to a blood sample concentration of each patient under various mixing conditions. This shows that since a large amount of protein nanoparticles are uniformly distributed at regular intervals in the three-dimensional porous hydrogel, there is little signal noise caused by non-specific bonding and each antibody is accurately detected with reproducibility.

### EXAMPLE 2

### Manufacturing of Protein Nanorod-Immobilized Hydrogel

(Manufacturing Expression Vector for Synthesis of Protein Nanorod Representing Disease Marker Detection Probe)

A production vector was manufactured by inserting a Sjögren's syndrome autoantibody detection probe (La protein) gene into a carboxyl terminal of a Saccharomyces cerevisiae-derived Sup35 protein known to be in the form of nanorods by self-assembly (FIG. 11(a)), and the production vector was expressed in E. coli so as to manufacture a water-soluble Sup35-La protein monomer (FIG. 11(b)).

Gene clones for coding NH₂-NdeI-hexahistidine-[Saccharomyces cerevisiae-derived Sup35 protein 1-61 (SEQ ID NO:6)]-G4S-BamHI-COOH and NH₂-BamHI-[human-derived La protein]-XhoI-COOH (or NH₂-BamHI-[human-derived La protein]-[biotin peptide]-XhoI-COOH) were PCR amplified by using an adequate primer and ligated to an NdeI-BamHI-XhoI cloning sites of pT7-7 and pET 28a, so that an expression vector pET28a-Sup35-La (or pT7-Sup35-La-biotin) for coding synthesis of a recombinant sup nanorod representing a disease marker detection probe on its surface was manufactured.

A nanorod assembly process was carried out by reference to the article “T. R. Serio, A. G et al., Science, 2000, 289, 1317-1321”.

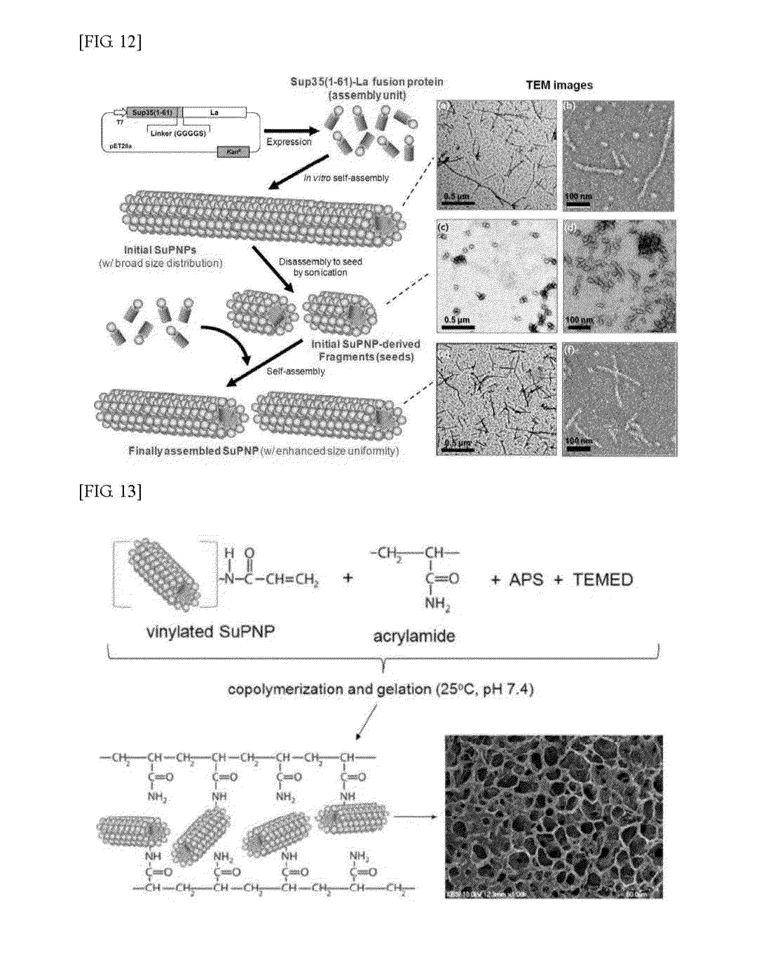

Referring to TEM images of FIG. 12, a Sup35-La protein expressed in the form of a monomer within E. coli was manufactured into protein particles (SuPNP) in the form of nanorods through an in-vitro assembly process. However, it was impossible to control the nanorods ranging in length from several ten nm up to 1 μm (FIGS. 12(a) and 12(b)). Therefore, Sup35-La protein seeds each having a diameter of 10 nm were manufactured through a disassembly process using a sonicator (FIGS. 12(c) and 12(d)), and then the Sup35-La protein monomers and the Sup35-La protein seeds were mixed at a ratio of 1:8, so that protein nanoparticles in the form of nanorods having uniform diameters of 100 to 400 nm were finally manufactured (FIGS. 12(e) and 12(f)).

(Manufacturing of Protein Nanorod-Immobilized Hydrogel)

10 mg of streptavidin and 0.01 mg of N-succinimidylacrylate (NSA) were incubated in a PBS buffer at 37° C. for 1 hour and bound to each other. Then, non-bound NSA was removed by ultrafiltration (Amicon Ultra 100K), so that streptavidin having a polymerizable chemical structure was finally manufactured. 30 μg of the streptavidin and 0.45% polyacrylamide (29:1 W/W acrylamide: bis-acrylamide) were mixed in the presence of 0.125% w/v ammonium persulfate (APS) and 0.125% w/v tetramethylethylenediamine (TEMED), and each 150 μl of the mixture was apportioned into a 96-well plate and polymerized at 25° C. for 16 hours, so that a streptavidin fusion hydrogel was manufactured. 10 μg of Sup35-La-biotin nanorods was incubated in the streptavidin fusion hydrogel for 1 hour so as to be immobilized thereto.

It was confirmed from SEM images of FIG. 13 that the protein nanorod fusion hydrogel had a three-dimensional structure having very uniform porosity.

(Sensitivity Examination of Protein Nanorod-Immobilized Hydrogel Fusion Material)

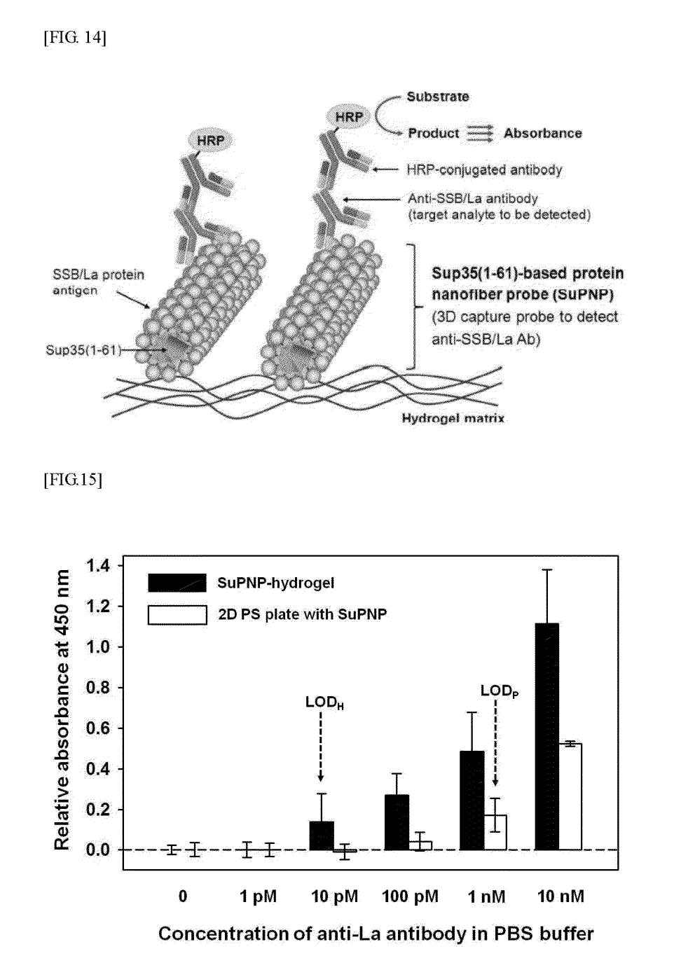

In order to evaluate the utility and performance of a three-dimensional protein nanorod fusion hydrogel diagnosis system, sensitivity analysis was carried out. The sensitivity analysis was carried out by using an anti-La antibody. As shown in FIG. 14, a hydrogel fused with a protein nanorod or a 2D PS surface was reacted with a sample (an anti-La antibody in a PBS buffer of different concentrations or an anti-La antibody in human blood), and then absorbance thereof was measured by using a secondary antibody bound to a HRP. Relative absorbance was obtained by deducting absorbance of a negative control group (with a concentration of 0) from the actually measured absorbance, and LOD_{H} and LOD_{P} denote LOD of SuPNP-hydrogel-based assay and LOD of 2D PS plate-based assay, respectively.

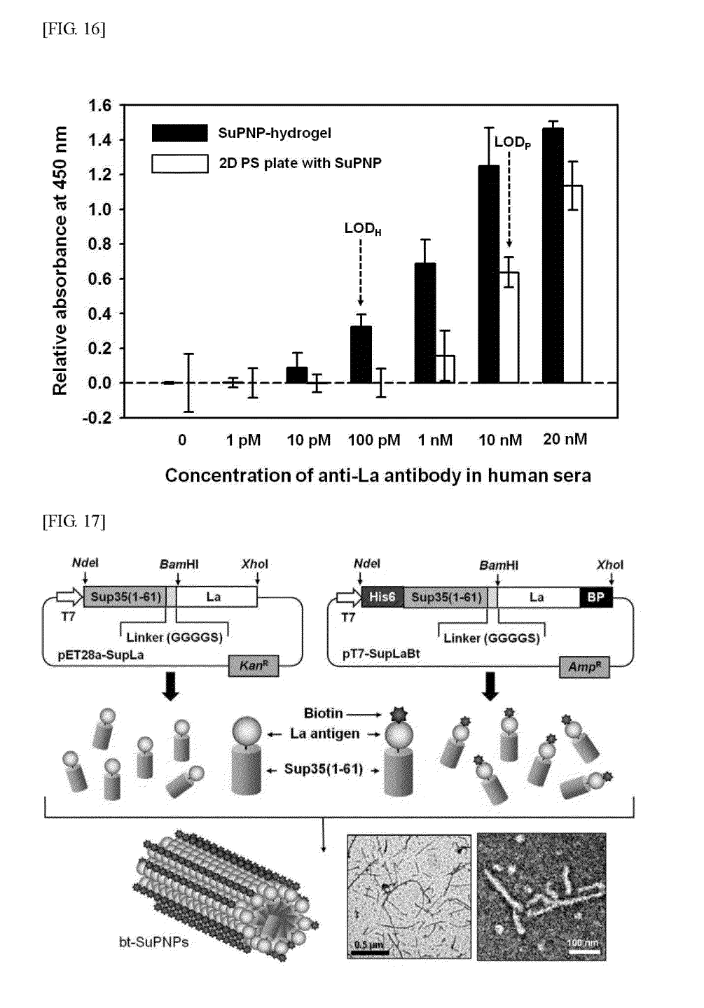

As shown in FIGS. 15 and 16, the SuPNP-hydrogel showed sensitivity 100 times higher than the two-dimensional plate.

A production vector was manufactured by inserting a biotin peptide into a carboxyl terminal of the sup35-La protein in order to immobilize the protein nanorod to the hydrogel by means of biotin-streptavidin bonding instead of covalent bond. After the production vector was expressed in E. coli, a sup35-La protein monomer was manufactured into protein nanorods (SuPNP) representing both biotin and a La protein through an in-vitro assembly process (FIG. 17).

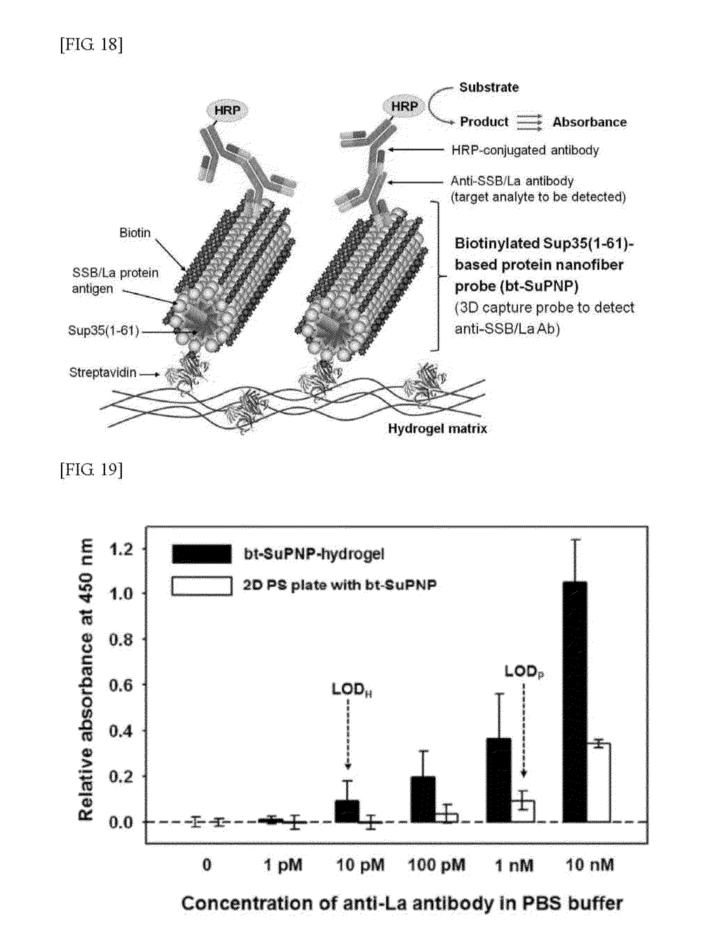

The protein nanorods representing both biotin and a La protein were immobilized to a hydrogel containing streptavidin by means of biotin-streptavidin bonding. In order to check utility and excellence of a three-dimensional biotin-streptavidin bond-based protein nanorod fusion hydrogel diagnosis system, a sensitivity analysis was carried out. The sensitivity analysis was carried out by using an anti-La antibody. As shown in FIG. 18, a hydrogel fused with a protein nanorod (bt-SuPNP) was reacted with a sample (an anti-La antibody in a PBS buffer or an anti-La antibody in human blood), and then absorbance thereof was measured by using a secondary antibody bound to a HRP.

As a result of a comparative analysis with a diagnosis system including protein nanorods immobilized to a two-dimensional polystyrene (PS) surface, the bt-SuPNP-hydrogel showed sensitivity 100 times higher than the two-dimensional diagnosis system (FIG. 19).

From the results described above, it can be seen that since protein nanoparticles were immobilized to a three-dimensional porous hydrogel in the present invention, a technical maturity level of a diagnosis system could be improved, and thus the present invention can be used as a base technology to be applied to diagnoses of other diseases.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A disease marker detection kit comprising:
a hydrogel to which a protein nanoparticle representing a disease marker detection probe is immobilized.

2. The disease marker detection kit of claim 1, wherein the protein nanoparticle representing a disease marker detection probe is manufactured from a chimeric protein fused with a protein capable of self-assembly and one or more disease marker detection probes.

3. The disease marker detection kit of claim 2, wherein the protein capable of self-assembly includes a ferritin heavy chain, a Sup35 protein derived from Saccharomyces cerevisiae, or a virus capsid protein.

4. The disease marker detection kit of claim 1, wherein the hydrogel to which a protein nanoparticle representing a disease marker detection probe is immobilized is manufactured by inducing a polymerization reaction between a protein nanoparticle expressed by Chemical Formula 1 below and a polymer precursor solution: 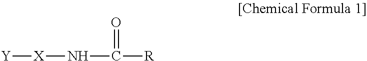
wherein in Chemical Formula 1,
X represents a protein nanoparticle,
Y represents a disease marker detection probe, and
R represents a vinyl group, an acryl group, or an acryl group substituted or not substituted by an alkyl having 1 to 30 carbon atoms.

5. The disease marker detection kit of claim 4, wherein the polymer includes one or more selected from the group consisting of polyacrylic acid, polyacrylamide, polyhydroxyethyl methacrylate, polyethyleneglycol, poly(N,N-ethylaminoethyl methacrylate), hyaluronic acid, and chitosan.

6. The disease marker detection kit of claim 4, wherein the polymer precursor solution further contains a polymerization initiator in an amount of 0.1 to 0.2 parts by weight with respect to 100 parts by weight of the polymer.

7. The disease marker detection kit of claim 6, wherein the polymerization initiator includes one or more selected from the group consisting of ammonium persulfate, tetramethylethyleneamine, riboflavin, riboflavin-5′-phosphate, 2-hydroxy-2-methylpropanon, and 2,2-diethoxyacetophenone.

8. The disease marker detection kit of claim 4, wherein the polymerization reaction is carried out by one or more methods selected from the group consisting of a chemical polymerization method, a UV polymerization method, and a photochemical polymerization method.

9. The disease marker detection kit of claim 1, wherein the disease marker includes an autoantibody of an autoimmune disease or an anti-virus antibody of a viral disease.

10. The disease marker detection kit of claim 1, wherein the disease marker detection probe includes an antigen protein specific to an autoantibody of an autoimmune disease including a human RO(SSA) protein or a human La(SSA) protein, or a virus-derived antigen protein including an HIV-1 gp41 peptide.

11. The disease marker detection kit of claim 1, further comprising:
a reporter probe configured to detect a bound form of the disease marker and the disease marker detection probe.

12. The disease marker detection kit of claim 11, wherein the reporter probe is any one of an anti-human IgG conjugated with a reporter enzyme including HRP (Horseradish Peroxidase) or AP (Alkaline Phosphatase); a virus antigen including an HIV-1 gp41 peptide; a biotin-bound virus antigen including a biotin-bound HIV-1 gp41 peptide; or a human autoantigen including a biotin-bound human La(SSA) protein or Ro(SSA) protein.

13. The disease marker detection kit of claim 11, wherein the reporter probe is labeled with a fluorescent material.

14. A disease marker detection method comprising:
reacting one or more hydrogels to which a protein nanoparticle representing a disease marker detection probe is immobilized with a sample to be detected;
reacting a reaction product obtained from the above step with a reporter probe; and
detecting one or more disease markers by measuring a change of absorbance or fluorescence intensity in the sample by a bound state of the disease marker-the disease marker detection probe-the reporter probe.

15. The disease marker detection method of claim 14, wherein the protein nanoparticle representing a disease marker detection probe is manufactured from a chimeric protein fused with a protein capable of self-assembly and one or more disease marker detection probes, and wherein the protein capable of self-assembly is selected from any one of a ferritin heavy chain, a Sup35 protein derived from Saccharomyces cerevisiae, or a virus capsid protein is used as the protein capable of self-assembly.

16. The disease marker detection method of claim 14, wherein the hydrogel to which a protein nanoparticle representing a disease marker detection probe is immobilized is manufactured by inducing a polymerization reaction between a protein nanoparticle expressed by Chemical Formula 1 below and a polymer precursor solution: 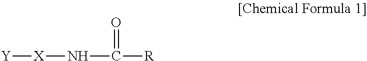
wherein in Chemical Formula 1,
X represents a protein nanoparticle,
Y represents a disease marker detection probe, and
R represents a vinyl group, an acryl group, or an acryl group substituted or not substituted by an alkyl having 1 to 30 carbon atoms.

17. The disease marker detection method of claim 16, wherein the polymer includes one or more selected from the group consisting of polyacrylic acid, polyacrylamide, polyhydroxyethyl methacrylate, polyethyleneglycol, poly(N,N-ethylaminoethyl methacrylate), hyaluronic acid, and chitosan.

18. The disease marker detection method of claim 14, wherein the disease marker includes an autoantibody of an autoimmune disease or an anti-virus antibody of a viral disease.

19. The disease marker detection method of claim 14, wherein the disease marker detection probe includes an antigen protein specific to an autoantibody of an autoimmune disease including a human RO(SSA) protein or a human La(SSA) protein, or a virus-derived antigen protein including an HIV-1 gp41 peptide.

20. The disease marker detection method of claim 14, wherein the reporter probe is any one of an anti-human IgG conjugated with a reporter enzyme including HRP (Horseradish Peroxidase) or AP (Alkaline Phosphatase); a virus antigen including an HIV-1 gp41 peptide; a biotin-bound virus antigen including a biotin-bound HIV-1 gp41 peptide; or a human autoantigen including a biotin-bound human La(SSA) protein or Ro(SSA) protein.
